(19) United States
(12) Patent Application Publication
(10) Pub. No.: US 2004/0250450 A1
(43) Pub. Date: Dec. 16, 2004

(54) Weighted shoe inserts and methods for use

(76) Inventors: Molly Snell, Vero Beach, FL (US); Kimberly Crawford, Vero Beach, FL (US)

Correspondence Address: FISH & NEAVE LLP, 1251 AVENUE OF THE AMERICAS, 50TH FLOOR, NEW YORK, NY, 10020-1105, US

(21) Appl. No.: 10/463,702

(22) Filed: Jun. 16, 2003

(51) Int. Cl. A43B 13/38; A61F 5/14; A43B 23/00; A43B 7/14

(52) U.S. Cl. 36/45; 36/140; 36/172

(57) ABSTRACT

A weighted orthotic insole placed in a standard shoe and used in a way to facilitate weight loss, strengthen several body muscles and provide cardiovascular conditioning. In addition to being removable, the orthotic insole is designed to maintain the support and comfort provided by the shoe in which it is used. In a preferred embodiment, the orthotic insole contains a first concentration of weight proximal to the heel portion of the shoe and a second, lighter concentration of weight proximal to the midsole portion of the shoe.

### BACKGROUND OF THE INVENTION

This invention relates to weighted inserts for use in shoes, and particularly to a weighted orthotic insole that may be inserted into a shoe in order to facilitate weight loss and improve one's overall health.

It is known that partaking in certain activities such as weight training, running and walking can be effective methods to induce weight loss and strengthen one's muscles.

The act of walking involves a sequence of motions of each limb known as the gait cycle. The gait cycle contains two phases: a stance phase during which the limb is in contact with the ground, and a swing phase during which the limb is lifted from the ground for advancement. The stance phase may be divided into three subphases known as the initial heel contact or the beginning stance, the full plantar or the midstance and the propulsion or the late stance subphases. Similarly, the swing phase may be divided into three subphases known as the initial swing, midswing and terminal swing subphases.

It is known to provide shoes or shoe inserts in order to solve orthopedic problems, provide comfort and protection to one's foot, and enhance certain phases or subphases of the gait cycle, particularly when performing activities such as walking.

However, it would be desirable to provide an apparatus and methods that can induce more effective weight loss when used in conjunction with a shoe, while providing a certain level of comfort and support during activities such as walking.

### SUMMARY OF THE INVENTION

It is an object of this invention to provide an orthotic insole that may be placed in a standard shoe and used in such a way to facilitate weight loss while walking.

It is another object of this invention to provide an orthotic insole that may be used for muscle toning and strengthening, cardiovascular conditioning and psychological endurance.

It is still another object of this invention to provide an orthotic insole that may be preformed or customized for an individual's foot in a way that can be convenient and comfortable to use.

In accordance with the present invention, there is provided an orthotic insole that is thin enough to fit into a standard shoe, and that contains weighted material distributed in a back portion and a more forward portion of the insole. Preferably, the orthotic insole is made of a polymer and contains a first concentration of weight proximal to the heel portion of the shoe and a second, lighter concentration of weight proximal to the midsole portion of the shoe. When used during normal ambulation, the weight concentrations increase the load applied to targeted muscles in the user's body, thereby promoting weight loss and improving the user's overall health.

### BRIEF DESCRIPTION OF THE DRAWINGS

The above and other advantages of the invention will be more apparent upon consideration of the following detailed description, taken in conjunction with the accompanying drawings, in which like reference characters refer to like parts throughout, and in which:

FIG. 1 is a top perspective view of a substrate according to the present invention;

FIG. 2 is a top perspective view of an orthotic insole comprising the substrate of FIG. 1;

FIG. 3 is a side cross-sectional view of the orthotic insole of FIG. 2, taken generally from line 3-3 of FIG. 2;

FIG. 4 is a bottom elevational view of the underside of the orthotic insole of FIG. 2, taken generally from line 4-4 of FIG. 2;

FIG. 5 is a top perspective view of a shoe incorporating the orthotic insole of FIG. 2;

FIG. 6 is a top perspective view of the shoe of FIG. 5 incorporating another embodiment of an orthotic insole according to the present invention;

FIG. 7A is a side view of a person engaging in the beginning stance subphase of the gait cycle according to the present invention;

FIG. 7B is a side view of a person engaging in the midstance subphase of the gait cycle according to the present invention;

FIG. 7C is a side view of a person engaging in the late stance subphase of the gait cycle according to the present invention;

FIG. 7D is a side view of a person engaging in the initial swing subphase of the gait cycle according to the present invention;

FIG. 7E is a side view of a person engaging in the midswing subphase of the gait cycle according to the present invention;

FIG. 7F is a side view of a person engaging in the terminal swing subphase of the gait cycle according to the present invention;

FIG. 8A is a front view of the muscle structure in a person's lower body that relates to the present invention; and

FIG. 8B is a back view of the muscle structure in a person's lower body that relates to the present invention.

### DETAILED DESCRIPTION OF THE INVENTION

The present invention provides a pair of weighted orthotic insoles that may be placed in standard shoes and used in a way to facilitate weight loss. In addition to being removable, each orthotic insole is designed to maintain the support and comfort provided by the shoe in which it is used.

In accordance with the invention, the orthotic insole may be made of polymer material that is weighted and may be covered with a soft orthotic cover. The orthotic insole may either be preformed or custom-molded for an individual's foot.

The polymer material used preferably is a polyphenylene sulfide compounded with glass fibers and mineral fillers. Alternatively, a dense polyurethane material may be used. Such material may or may not contain metal powder or pellets. The metal preferably includes lead.

The orthotic cover preferably is a polyvinal acetate emulsion. Alternatively, the insole may be covered with moleskin, felt, rayon, vinyl, silicon, foam or any such material.

The orthotic insole preferably contains two main concentrations of weight. The first concentration is located in a portion designed to come in contact with the heel portion of a shoe. The second concentration of weight is located in a portion designed to come in contact with a more forward portion of a shoe, such as the midsole portion.

The portion of the orthotic insole containing the first concentration of weight preferably is thicker and larger in volume than the portion of the insole containing the second concentration of weight. The portion containing the first concentration of weight is also preferably heavier than the portion containing the second concentration of weight.

As stated above, the gait cycle contains a stance phase and a swing phase. Approximately 60% of gait time is spent in the stance phase, with both legs being in contact with the ground and supporting the upper body in the beginning 10% and the ending 10% of that phase. The other 40% of gait time is spent in the swing phase, during which one leg is lifted to achieve forward advancement. Different body muscles are activated during different phases and subphases of the gait cycle, thereby resulting in the loading of particular groups of muscles at different times.

A pair of orthotic insoles as disclosed herein maximally exercises different muscles and muscle groups during the stance phase, while protecting and preventing injury of muscles and joints during the swing phase. This results in increased biomechanical work to targeted muscles of the thigh, buttocks, calves and back. The increased work also increases the heart rate and metabolic rate. Therefore, the orthotic insoles promote the growth of muscle fibers and elevate the cardiovascular response, thereby strengthening and toning the targeted muscles, increasing the lean-to-fat body ratio and reducing the risk of diseases associated with obesity.

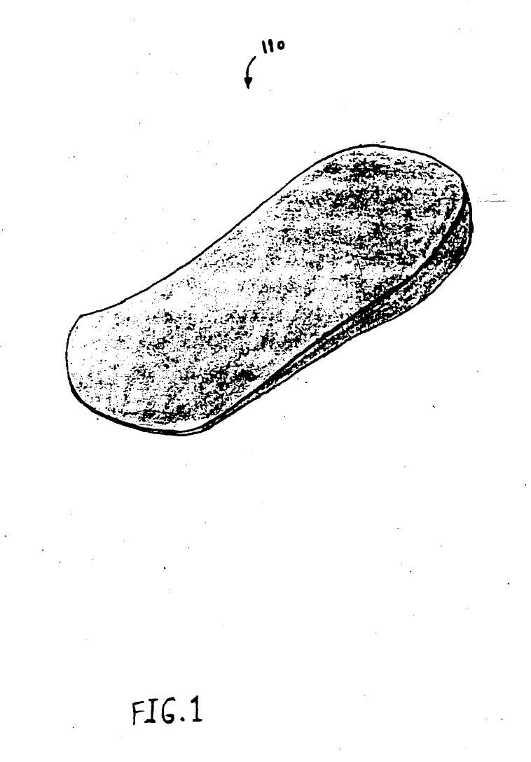

The invention will now be described with reference to FIGS. 1-8.

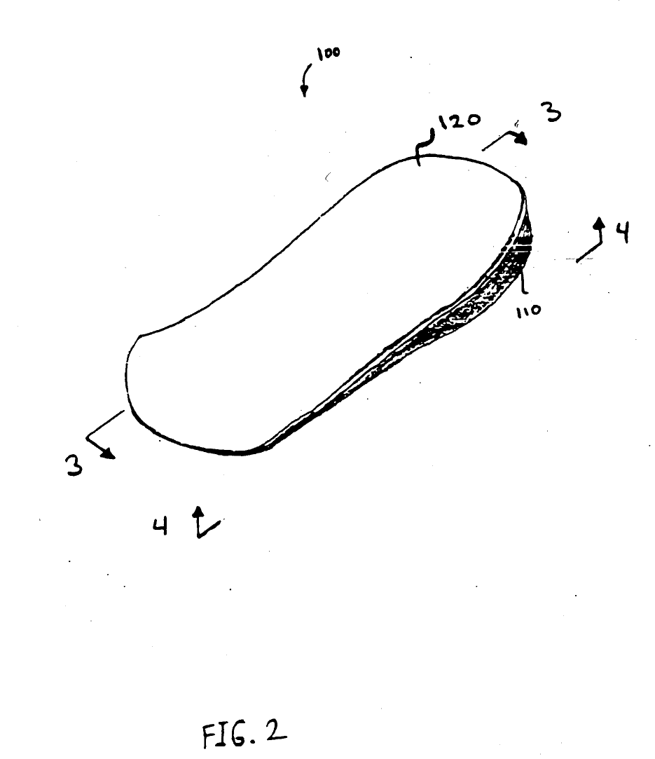

FIG. 1 shows an embodiment of an orthotic substrate 110 that may be used in orthotic insole 100 shown in FIG. 2, according to the present invention. Orthotic insole 100 may be placed in a shoe, as shown in FIG. 5, in such a way that its top portion constitutes a stepping surface that comes in contact with the foot, while its bottom surface comes in contact with the shoe's sole.

In a preferred embodiment, substrate 110 may be made of polyphenylene sulfide, which is available under the trademark RYTON. Substrate 110 may also contain lead, and may weigh 1 pound or more. The polyphenylene sulfide and lead combination may be formed through injection-molding, or any other process.

In an alternative embodiment, substrate 110 may be made of another polymer such as polyurethane, or any other relatively dense material. Substrate 110 may or may not be injected with metal powder, metal pellets, non-mineral filler or any other filling material to increase its weight.

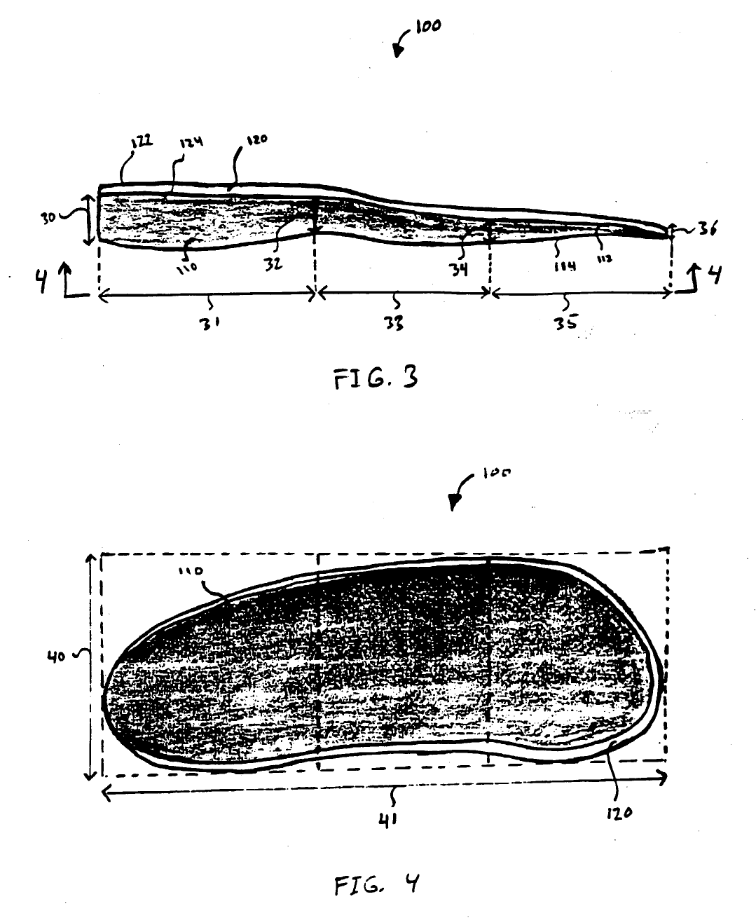

Substrate 110 may be covered with a soft layer. Preferably, cover 120 may cover substrate 110 from the top as shown in FIGS. 2 and 3. Substrate 110 may have top surface 112 and bottom surface 114. Cover 120 may have outer surface 122 and inner surface 124. Cover 120 may be placed on or may cover substrate 110 such that top surface 112 of substrate 110 and inner surface 124 of cover 120 are in contact with one another. Cover 120 may extend beyond the front and the sides of substrate 110 as shown in FIG. 4. Cover 120 may extend beyond the front, sides or the back of substrate 110, or any combination of the same, such that cover 120 may be trimmed to better fit in a shoe.

In a preferred embodiment, cover 120 may be made of soft material that provides cushioning such as polyvinal acetate, adding to the comfort provided by insole 100. Such material is available under the trademark VINAR. Alternatively, cover 120 may be made of moleskin, felt, rayon, vinyl, silicon, foam or any combination of the same.

As shown in FIG. 3, the back portion of substrate 110, which may be enclosed between segments 30 and 32 and may extend along segment 31, may contain a first concentration of weight. The middle portion of substrate 110, which may be enclosed between segments 32 and 34 and may extend along segment 33, may contain a second concentration of weight. The front portion of substrate 110, which may be enclosed between segments 34 and 36 and may extend along segment 35, may contain a third concentration of weight.

The first concentration located in the back portion of substrate 110 preferably is designed to align with the heel, or the heel portion of a shoe, when orthotic insole 100 is used in a shoe. The second concentration of weight located in the middle portion of substrate 110 preferably is designed to align with the midsole, or the midsole portion of a shoe, when orthotic insole 100 is used in a shoe.

In accordance with the embodiment illustrated in FIGS. 3 and 4, the thickness of the back portion of substrate 110 preferably tapers generally from a first thickness, corresponding to the length of segment 30, at the back end of insole 100, to a second thickness, corresponding to the length of segment 32. The back portion has an approximate length corresponding to the length of segment 31.

Similarly, the thickness of the middle portion of substrate 110 preferably tapers generally from the second thickness, corresponding to the length of segment 32, to a third thickness, corresponding to the length of segment 34. The middle portion has an approximate length corresponding to the length of segment 33.

The thickness of the front portion of substrate 110 preferably tapers off generally from the third thickness, corresponding to the length of segment 34, to the front end of insole 100. At the front end of insole 100, the thickness of insole 100 preferably is equal to the thickness of cover 120, corresponding to the length of segment 36. The front portion has an approximate length corresponding to the length of segment 35.

Orthotic insole 100 has a predominant width corresponding to the length of segment 40, and a general length corresponding to the length of segment 41.

As can be seen, the back portion of substrate 110 may be thicker than its middle and front portions. Preferably, substrate 110 tapers generally from its back end to its front end such that the volume of substrate 110 generally decreases from its back end to its front end. As a result, the weight of substrate 110 decreases from its back end to its front end, thereby creating the different concentrations of weight in substrate 110. In such an embodiment, the density of material used throughout substrate 110 is substantially uniform.

Alternatively, the different concentrations of weight may be created through the process of injecting or placing weighted material in the back portion of substrate 110 to form the first concentration of weight. Additionally, weighted material may be injected or placed in the middle portion of substrate 110 to form the second concentration of weight, or, alternatively, in the front portion of substrate 110 to form the third concentration of weight.

Regardless of how the different concentrations of weight are formed, the back portion of substrate 110 is preferably heavier than the middle portion of substrate 110. The back portion of substrate 110 is also preferably heavier than the front portion of substrate 110. In a preferred embodiment where a first concentration of weight and a second concentration of weight are provided, the first concentration of weight weighs between approximately 40% and approximately 75% more than the second concentration of weight. Together, the first and second concentrations of weight may account for most of the weight of orthotic insole 100.

Segments 30-36 of FIG. 3 and segments 40-41 of FIG. 4 may be of any lengths, provided that insole 100 may fit in a shoe and have a weight distribution that is heavier in the back portion of the insole than in the remaining portions. Insole 100 may be custom-molded for an individual's foot, or, alternatively, insole 100 may be preformed or designed to fit in shoes of various sizes.

For example, in an embodiment of insole 100 that is designed to be used with an approximate size-8 shoe for women, the orthotic insole 100 is approximately 17 cm long an predominately 6 cm wide. The back portion of substrate 110 is approximately 7 cm long, and tapers in thickness from approximately 1.2 cm to approximately 1 cm. The middle portion of substrate 110 is approximately 6 cm long and tapers in thickness from approximately 1 cm to approximately 0.5 cm, while the front portion is approximately 4 cm long. The thickness of cover 120 may be approximately 0.1 cm.

Together, substrate 110 and cover 120 form orthotic insole 100, which may weigh approximately 1 pound, 12 ounces (1.75 lbs). The ratio of polymer material to filling material, the thickness of substrate 110 or both, may be adjusted as the size of insole 100 is varied.

As shown in FIG. 5, orthotic insole 100 may be placed in shoe 500. A pair of orthotic insoles may be used in walking shoes, hiking shoes, regular shoes or any athletic or other standard shoes. Orthotic insole 100 may be inserted into and removed from shoe 500 without altering shoe 500 in any way. As seen in FIG. 5, orthotic insole 100 does not extend along the entire length of sole 510 of shoe 500.

However, in another embodiment shown in FIG. 6, orthotic insole 600 may include an additional portion 660, such that insole 600 extends along the entire length of sole 510 of shoe 500. Such an insole may be inserted into and removed from shoe 500 without altering shoe 500 in any way, as shown in FIG. 6.

Insole 600 may include a substrate and a cover. Portion 660 may be made of the same material as orthotic insole 100, without injecting or placing any mineral fillers or weighted material in it. Portion 660 may be molded with, or as part of, the substrate, and the cover of insole 600 may extend along the entire length of the substrate. Portion 600 may be made of any material such that the weight of portion 660 is substantially less than that of the remainder of insole 600 and does not affect the weight balance of a person using insole 600.

Alternatively, and in another preferred embodiment, the cover of insole 600 may extend beyond the front portion of the substrate such that portion 660 constitutes part of the cover. In such an embodiment, insole 600 may or may not extend along the entire length of sole 510. Similarly, the cover of insole 600 may extend beyond the back portion of the substrate.

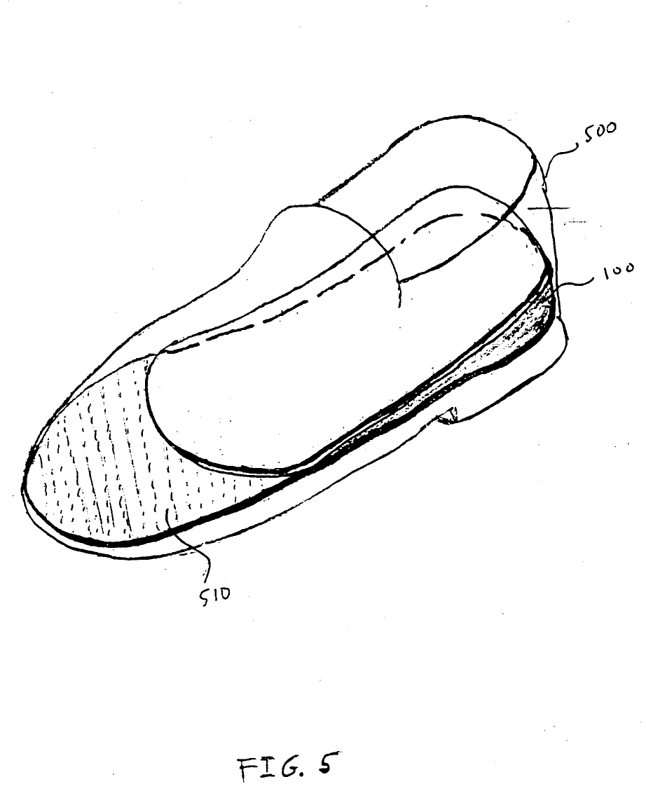

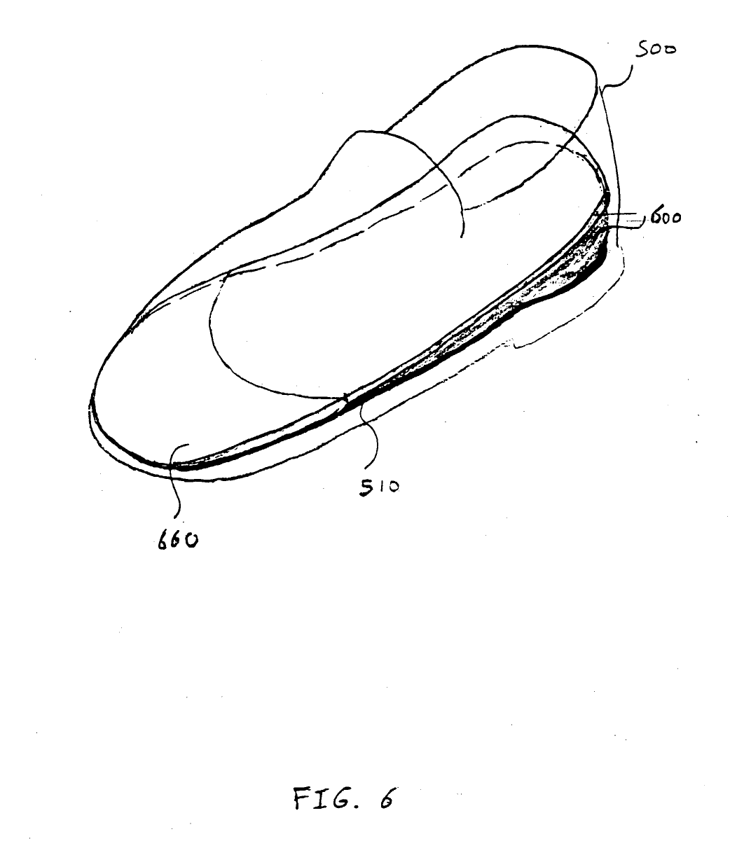

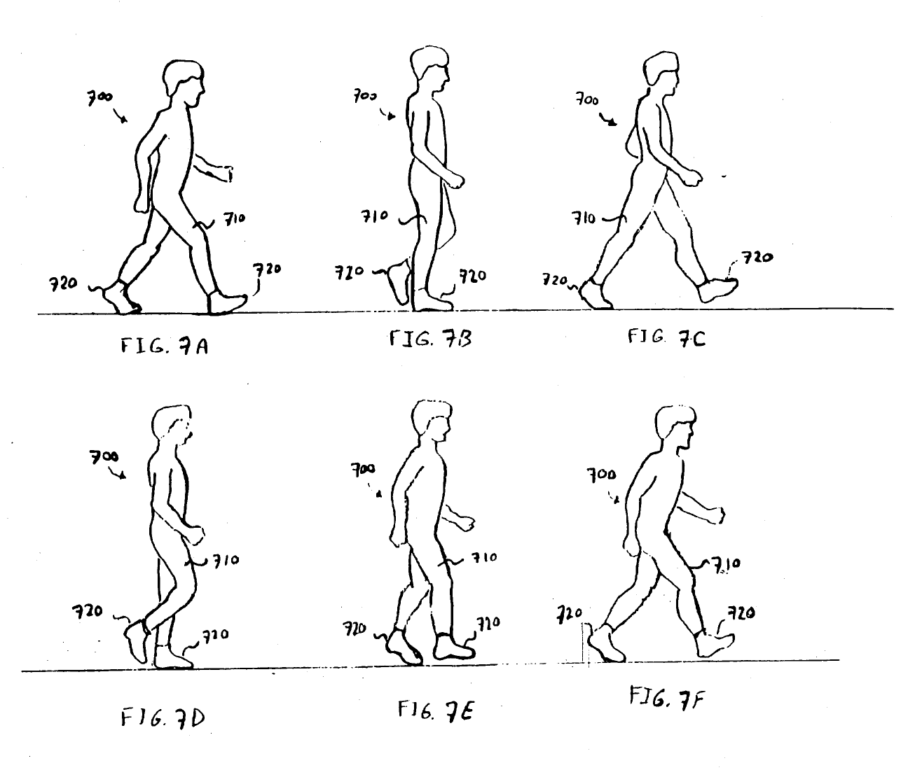

FIGS. 7A-7F depict a sequence of functions in which a person 700 wearing shoes 720 is engaging during the different phases of a single gait cycle. It is assumed that each one of shoes 720 includes an orthotic insole such as the one described in accordance with the present invention. FIGS. 7A-7C show the sequence of functions by right leg 710 of person 700 associated with the stance phase of the gait cycle. FIGS. 7D-7F show the sequence of functions by right leg 710 of person 700 associated with the swing phase of the gait cycle.

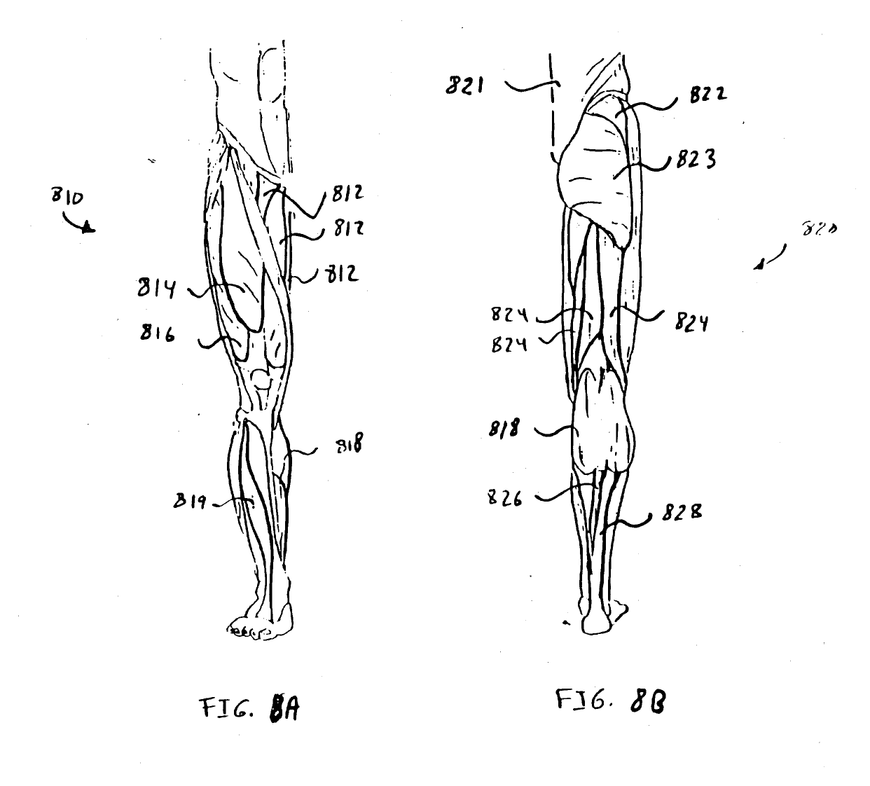

FIGS. 8A and 8B show the various muscles that are targeted by the present invention during the different phases of the gait cycle. FIGS. 7A-7F will now be described in conjunction with FIGS. 8A and 8B.

With respect to FIG. 7A, person 700 is in the beginning stance subphase as leg 710 initially contacts the ground. The concentration of weight in the back portion of the insole placed in shoe 720 coupled to leg 710 loads the group of muscles that are activated during this subphase. Such a group preferably includes the largest muscles that are activated, such as the gluteus maximus muscle 823, the gluteus medius muscle 822, the hamstring muscles 824, the rectus femoris muscle 814 and the vastus lateralis muscle 816. Such loading increases the biomechanical work exerted by these muscles. Additionally, the concentrations of weight in the back and the more forward portions of the insole targets the erector spinae muscles 821, both strengthening them and protecting them from injury.

With respect to FIG. 7B, person 700 is in the midstance .subphase as weight is transferred to leg 710, which is in full contact with the ground. The lighter concentration of weight in the more forward portion of the insole placed in shoe 720 coupled to leg 710 loads the group of muscles that are activated during this subphase. Such a group preferably includes the smaller muscles that are activated and targeted by the more appropriate, smaller concentration of weight. Such muscles include the gastrocnemius muscle 818, the soleus muscle 828 and the tibialis posterior muscle 826. Similarly, such loading increases the biomechanical work exerted by these muscles.

With respect to FIG. 7C, person 700 is in the late stance subphase as leg 710 prepares to swing and lift from the ground. The lighter concentration of weight in the more forward portion of the insole placed in shoe 720 coupled to leg 710 loads the group of muscles that are activated during this subphase. Such a group preferably includes the adductor muscles 812, which exert more biomechanical work when loaded by the concentration of weight in the more forward portion of the insole.

With respect to FIG. 7D, person 700 is in the initial swing subphase as leg 710 is lifted from the ground and swings forward. With respect to FIG. 7E, person 700 is in the midswing subphase as leg 710 continues to advance forward such that it is relatively perpendicular to the ground. With respect to FIG. 7F, person 700 is in the terminal swing subphase as leg 710 prepares to come in contact with the ground prior to the beginning stance phase described in conjunction with FIG. 7A.

The distribution of heavier to lighter weight concentrations in the back and more forward portions of the insole placed in shoe 720 is believed to play a role in minimizing the weight applied to select areas during all three subphases of the swing phase shown in FIGS. 7D-7F. Such areas preferably include the ankle and the tibialis anterior muscle 819, which is activated during the entire swing phase. In addition, the diminished back to front weighting in the insole prevents overloading of the knees, hips and back during ambulation.

Therefore, by placing a pair of orthotic insoles described herein in his or her shoes, in accordance with the present invention, a person may increase the work done by his or her thighs, buttocks, calves and back muscles while partaking in an exercise such as walking. As a result, the person's metabolic rate and cardiovascular response are increased while his or her muscles are toned and strengthened. This, in turn, facilitates weight loss and increases the lean-to-fat body ratio. At the same time, the person's joints and muscles may be protected from injury.

Also, frequent exercising using the orthotic insoles described herein may reduce the risk of ailments and diseases associated with obesity, such as high blood pressure, coronary heart disease, osteoarthritis, type 2 diabetes, sleep apnea and even breast and colon cancer. Finally, frequent exercising using the orthotic insoles promotes psychological endurance which safeguards against somatic physical ailments, enhances self esteem and decreases the incidence of anxiety and depression.

Thus it is seen that a weighted insole for use in a shoe for facilitating weight loss and improving one's overall health has been provided. One skilled in the art will appreciate that the present invention can be practiced by other than the described embodiments, which are presented for purposes of illustration and not of limitation, and the present invention is limited only by the claims which follow.

What is claimed is:

1. A removable insole for use in an athletic shoe, the shoe having a heel portion and a forward portion, the insole comprising:
a first portion proximal to the heel portion, the first portion having a first concentration of weight; and
a second portion integrally joined with the first portion such that the second portion is proximal to the forward portion, the second portion having a second concentration of weight; wherein:
the second concentration of weight weighs less than the first concentration of weight.

2. The removable insole of claim 1 wherein the first concentration of weight weighs between approximately 40% and approximately 75% more than the second concentration of weight.

3. The removable insole of claim 1 wherein at least one of the first and second concentrations of weight comprises filling material.

4. The removable insole of claim 3 wherein the filling material comprises lead.

5. The removable insole of claim 3 further comprising a polymer material in which the filling material is embedded.

6. The removable insole of claim 5 wherein the polymer material comprises polyphenylene sulfide.

7. The removable insole of claim 5 wherein the polymer material comprises polyurethane.

8. The removable insole of claim 1 further comprising a cover that covers the first and second portions.

9. The removable insole of claim 8 wherein the cover comprises a material that is selected from the group consisting of polyvinal acetate, moleskin, felt, rayon, vinyl, silicon, and foam.

10. The removable insole of claim 1 wherein the first portion comprises:
a first end distal from the second portion, the first end having a first thickness; and
a second end proximal to the second portion, the second end having a second thickness that is smaller than the first thickness.

11. The removable insole of claim 10 wherein the forward portion comprises a midsole portion, the second portion comprising:
a first end proximal to the first portion, the first end having substantially the same thickness as the second thickness of the first portion; and
a second end distal from the first portion, the second end having a third thickness that is smaller than the second thickness of the first portion.

12. The removable insole of claim 11 further comprising a third portion integrally joined to the second end of the second portion, the third portion comprising:
a first end proximal to the second portion, the first end having substantially the same thickness as the third thickness of the second portion; and
a second end distal from the second portion, the second end having a fourth thickness that is smaller than the third thickness of the second portion.

13. An orthotic insole for use in a shoe, the shoe having a heel portion and a forward portion, the orthotic insole comprising a polymer substrate comprising:
a first concentration of weight proximal to the heel portion; and
a second concentration of weight proximal to the forward portion, the second concentration of weight weighing less than the first concentration of weight.

14. The orthotic insole of claim 13 wherein the first concentration of weight weighs between approximately 40% and approximately 75% more than the second concentration of weight.

15. The orthotic insole of claim 13 wherein the polymer substrate comprises polyphenylene sulfide.

16. The orthotic insole of claim 13 wherein the polymer substrate comprises polyurethane.

17. The orthotic insole of claim 13 wherein at least one of the first and second concentrations of weight comprises filling material.

18. The orthotic insole of claim 17 wherein the filling material comprises lead.

19. The orthotic insole of claim 13 further comprising an orthotic cover that covers the polymer substrate.

20. The orthotic insole of claim 19 wherein the orthotic cover comprises a material that is selected from the group consisting of polyvinal acetate, moleskin, felt, rayon, vinyl, silicon, and foam.

21. The orthotic insole of claim 13 wherein the polymer substrate comprises a first end proximal to the heel portion and a second end distal from the heel portion such that the thickness of the polymer substrate generally tapers from the first end to the second end.

22. A method for inducing weight loss in a person using the removable insole of claim 1 in a shoe, the method comprising:
loading a first group of the person's muscles with the first concentration of weight while the shoe is in contact with the ground during ambulation; and
loading a second group of the person's muscles with the second concentration of weight while the shoe is in contact with the ground during ambulation, the second group of muscles including muscles that are generally smaller than the muscles in the first group of muscles.

23. A method for using an insole in a person's shoe, the shoe having a heel portion and a forward portion, the insole having a first concentration of weight proximal to the heel portion and a second concentration of weight proximal to the forward portion, the second concentration of weight weighing less than the first concentration of weight, the method comprising:
loading a first group of the person's muscles with the first concentration of weight while the person's shoe is in contact with the ground during ambulation; and
loading a second group of the person's muscles with the second concentration of weight while the person's shoe is in contact with the ground during ambulation, the second group of muscles including muscles that are generally smaller than the muscles in the first group of muscles.

24. The method of claim 23 further comprising minimizing a load applied to select areas in the person's body by selecting a preferred ratio of first concentration of weight to second concentration of weight.

25. A method for using the orthotic insole of claim 13 in a shoe worn on a person's foot, the method comprising:
inserting the orthotic insole in the shoe;
inserting the foot in the shoe;
planting a leg associated with the foot on the ground such that a plurality of the person's muscles are loaded with the first and second concentrations of weight while the shoe is in contact with the ground;
swinging the leg such that a load applied to select areas in the person's body is minimized while the shoe is not in contact with the ground; and
repeating the steps of planting the leg and swinging the leg.
